(19) United States
(12) Patent Application Publication
(10) Pub. No.: US 2013/0040894 A1
(43) Pub. Date: Feb. 14, 2013

(54) INHIBITION OR ACTIVATION OF SERINE/THREONINE ULK3 KINASE ACTIVITY

(71) Applicants: Priit Kogerman, Tabasalu (EE); Alla Maloverjan, Tallinn (EE); Marko Piirsoo, Tallinn (EE); Piret Michelson, Harjumaa (EE); Torben Osterlund, Lund (SE)

(72) Inventors: Priit Kogerman, Tabasalu (EE); Alla Maloverjan, Tallinn (EE); Marko Piirsoo, Tallinn (EE); Piret Michelson, Harjumaa (EE); Torben Osterlund, Lund (SE)

(73) Assignee: TALLINN UNIVERSITY OF TECHNOLOGY, Tallinn (EE)

(21) Appl. No.: 13/500,902

(22) PCT Filed: Oct. 6, 2010
(86) PCT No.: PCT/EE10/00016

§ 371 Date: Jul. 16, 2012

Related U.S. Application Data
(60) Provisional application No. 61/278,378, filed on Oct. 6, 2009.

(51) Int. Cl. C12N 9/99; C12N 5/076; C12N 5/09; C07K 2/00; A61P 19/00; A61K 38/02; A61P 35/00; A61K 31/7088; A61P 15/08; A61P 17/14; C12N 5/071; C07K 7/08

(52) U.S. Cl. 514/19.4; 435/455; 435/375; 435/184; 530/300; 530/326; 514/1.1; 514/19.3; 514/19.5; 514/44 R

(57) ABSTRACT

The present invention relates to human serine/threonine kinase ULK3 and its ability to regulate GLI transcription factors; mediators of SHH signaling. This disclosure demonstrates that ULK3 enhances endogenous and over-expressed GLI1 and GLI2 transcriptional activity in cultured cells, and ULK3 alters subcellular localization of GLI1. According to this disclosure ULK3 is an autophosphorylated kinase and phosphorylates GLI proteins in vitro. A peptide sequence in GLI1 C-terminus that is phosphorylated by ULK3 is provided in this disclosure. ULK3 catalytical activity is shown to be crucial for its function in SHH pathway. This disclosure shows that serine/threonine kinase ULK3 is involved in the SHH pathway as a positive regulator of GLI proteins. Furthermore, a therapeutic method in SHH dependent human disorders is disclosed by pharmacological inhibition of ULK3 kinase activity. Identification of ULK3 substrate sequence in GLI1 allows the design of peptide-based modulators of its kinase activity.

### TECHNICAL FIELD

The present invention relates to novel molecules, such as proteins, polypeptides and nucleotides, involved in the transduction of signals in the hedgehog (Hh) pathway, which takes place during the development of the cells of a human body. The invention also relates to certain advantageous uses of the molecules according to the invention in diagnosis and therapy.

### BACKGROUND OF THE INVENTION

Hedgehog (Hh) pathway is involved in numerous biological processes during embryonic development of many animals ranging from fruit fly to mammals [1]. During postnatal life Hh signaling contributes to tissue homeostasis maintenance and controls neurogenesis and stem cell behavior. In humans aberrant activation of Hh signaling is associated with various developmental abnormalities and several types of cancer (reviewed in [2]). In spite of comprehensive studies, many gaps still exist in understanding the intracellular events initiated by Hh proteins.

Although Hh signaling seems to be conserved between invertebrates and vertebrates in many aspects, there are principal differences among species in intracellular interpretation of Hh signal ([3, 4]). In Drosophila Hh pathway is mediated through transcription factor Cubitus interruptus (Ci) that comprises both activator and repressor functions. In vertebrates the function of Ci is divided between three homologous proteins, Gli1, Gli2 and Gli3. Gli1 is an obligatory activator, Gli2 and Gli3 carry activator or repressor functions, with Gli3 being the strongest repressor. In the absence of Hh, Gli1 is generally not expressed; Gli2 and Gli3 proteins (as Ci in Drosophila) are mostly present in a C-terminally processed transcriptional repressor form, and full-length activator forms are tethered in the cytoplasm or subjected to proteosomal degradation [5]. The signaling is initiated through binding of Hh proteins (Sonic, Desert or Indian in vertebrates) to the 12-pass membrane receptor Patched (Ptch). Binding of ligand allows another transmembrane protein, Smoothened (Smo), to be relieved from the inhibitory effect of Ptch. Through its carboxyl cytotail Smo triggers the intracellular signaling cascade that culminates in activation, stabilization and nuclear translocation of Ci/Gli transcriptional activator forms. In the nucleus full-length Gli proteins are able to activate expression of the target genes, for instance Ptch and Gli1 (reviewed in [1] and [6]).

Most of the signal transduction events are mediated by protein kinases. Several kinases are shown to be involved in Hh signaling pathway and regulating Ci/Gli activity. Some kinases regulate negatively the pathway in the absence of Hh and exert positive effects in the presence of Hh ligands. Serine/threonine kinases Fused (Fu), PKA, GSK3, CK1, PI3K, Akt, PKCδ, MEK1, ERK1, MAP3K10 and tyrosine kinases DYRK1 and DYRK2 have been reported to affect Ci- and/or Gli-dependent Hh signaling ([7-15]). However, not all kinases have been found to be functionally conserved between vertebrates and invertebrates. Serine/threonine kinase Fu is, perhaps, one of the most puzzling molecules in Hh signaling.

Drosophila genetic and biochemical studies ascertain Fu (dFu) as a component of Hh signaling [8, 12]. dFu is essential for the embryonic development as homozygous dFu mutants are not viable. Partial loss of dFu activity in Drosophila results in variety of phenotypes including a fusion of longitudinal wing veins 3 and 4 that characterizes perturbation of Hh signalling [8, 16, 17]. The predominant function of dFu is to counteract with Suppressor of Fused (dSufu), known as a cytoplasmic inhibitor of Ci [16]. dFu is able to bind directly to kinesin-like protein Costal-2 (Cos2), dSmo and dSufu [18-20]. According to the accepted model, in the absence of Hh those proteins down-regulate the pathway. dSmo, Cos2, dFu, Ci and, probably, dSufu form a complex that tethers full-length Ci in the cytoplasm preventing its nuclear localization. Besides that, the complex interacts with PKA, Shaggy (Drosophila homologue of GSK3) and CK1 through Cos2. These protein kinases are responsible for proteolytic cleavage of Ci in resting cells and phosphorylation followed by subsequent activation of dSmo C-terminus in response to Hh. Activation of the pathway also induces phosphorylation of dFu, dSufu and Cos2, whereas phosphorylation of dSmo, Cos2 and dSufu depends on dFu kinase activity [21-25]. Thus, the kinase activity of dFu is essential for the generation of Ci transcriptional activator form in the presence of Hh ligand.

Until now, one mammalian orthologue of dFu has been reported ([26, 27]. Human serine/threonine kinase STK36 (also known as FUSED) has been identified as a protein sharing the highest homology with dFu (27% of overall identity and 51% of identity in kinase domain). Human and mouse Fu homologues (hFU and mFu, respectively) have been shown to participate in mediating GLI-dependent Hh signaling in vitro, but in contrast to dFu, independently of the functional kinase domain [26, 28]. Genetic studies have shown that hFU, over expressed in fu mutant flies, cannot rescue their phenotype [29]. Besides that, contrary to dFu, mFu is dispensable for embryonic development [30, 31]. However, it seems to be highly important later in development, as newborn mFu^{-/-} mice display extensive brain defects and die within 3 weeks after birth [31]. Thus, the role of mammalian Fu in Hh signaling appears to differ from that of dFu, suggesting that other or additional kinases are involved in the regulation of Gli activity.

### SUMMARY OF THE INVENTION

The instant invention relates to serine/threonine kinase capable of phosphorylating GLI proteins and promoting nuclear localization of GLI1. The instant invention accordingly relates to methods to activate/deactivate Hh signaling via translocation of GLI proteins.

In one aspect, the invention provides a method to activate Hh signaling pathway in mammalian cells by transfecting the cells with a vector comprising an isolated nucleic acid sequence encoding serine/threonine kinase of SEQ ID NO: 14.

In another aspect, the invention provides a method to interfere with conditions that are related to Hh signaling pathway by directing activation of the pathway to specified cells. Conditions to be treated include conditions such as hair loss, male infertility, and dwarfism.

In one aspect, the invention identifies a novel peptide sequence (SEQ ID NO: 15) that includes four potential phosphorylation sites for ULK3 serine/threonine kinase.

In still another aspect, the invention provides a method to inhibit Hh signaling pathway by providing a molecule inhibiting the serine/threonine kinase activity of ULK3 protein.

According to yet another aspect of the invention, the inhibitor molecule binds the ATP binding site and/or the protein/peptide substrate binding site of the kinase.

According to a further aspect of the invention, the inhibitor molecule binds to the hydrophilic region in the C-terminal non-kinase domain of ULK3.

According to one aspect of the invention, the inhibitor molecule is a pseudosubstrate designed based on SEQ ID NO:15.

According to one aspect of the invention, a high affinity inhibitor of ULK3 is designed based on SEQ ID NO:1 or SEQ IND NO:15.

According to yet another aspect the inhibitor may be a multifunctional inhibitor containing both the active site binding moiety and the hydrophilic region-binding moiety covalently connected to each other.

According to one aspect of the invention the inhibitor is cell permeable drug molecule that interferes with the ULK3 regulatory, function in the Hh pathway.

The present disclosure provides the cloning of human serine/threonine kinase ULK3 that has been annotated as belonging to unc-51-like family of serine/threonine kinases, but shares similarity with STK36 and dFu proteins. This disclosure shows that ULK3 is an autophosphorylated kinase. In cultured cells ULK3 is able to enhance endogenous and overexpressed GLI1 and GLI2 transcriptional activity and to induce nuclear translocation of GLI1. This disclosure shows that ULK3 phosphorylates GLI proteins in vitro, and GLI1 has at least two phosphorylation sites situated in N- and C-terminus of the protein. This disclosure also identifies the phosphorylated peptide sequence in the C-terminal end of GLI1 protein.

This disclosure further shows that in contrast to STK36, the kinase-deficient mutants of ULK3 are inactive indicating that functional kinase domain of ULK3 is required for the regulation of GLI protein activity. Also this disclosure shows that ULK3 expression is higher in fetal brain and in a number of postnatal tissues where Hh signaling is known to be active. This disclosure provides that ULK3 is involved in Hh pathway as a positive regulator of Gli proteins. Accordingly human ULK3 is identified as a serine/threonine kinase regulating positively Hh pathway in mammalian cells.

### BRIEF DESCRIPTION OF THE DRAWINGS

FIG. 1 depicts the structure, phylogenetic analysis and expression of human ULK3

A. Phylogram tree of unc51 and fused subfamilies of proteins.

The sequences of full-length proteins of fused and unc51 subfamilies were obtained from NCBI database and subjected to multiple sequence alignment using ClustalW program (EMBL-EBI). The phylogram tree was built using the algorithm based on GONNET 250 matrix. According to the calculated distances between the proteins, ULK3 belongs to the STK36/fused subfamily. The same result was obtained using BLOSUM 30 matrix.

B. Structure of ULK3 gene and the expressed ULK3 protein.

The ULK3 gene is 7 kb, the coding part is 1419nt. The ULK3 gene contains 16 exons whereas first 7 exons code the putative kinase domain. The point mutations changing lysine nr 44 and 139 to arginine were generated in exons 2 and 4 in positions 131 and 416, respectively (the numbers are given according to the translation initiation codon ATG). ULK3 protein is 472 amino acids (aa). It is a putative serine-threonine kinase with amino-terminal 270 aa kinase domain. Both mutated lysines are highly conserved and belong to the functional domains of kinases—lysine nr 44 belongs to the ATP-binding pocket and the lysine nr 139 is in catalytic loop and substrate binding pocket (blastp conserved domain analysis, NCBI).

C. Quantitative RT-PCR analysis of ULK3 mRNA expression in human tissues.

The data was normalized by HPRT mRNA levels and is shown relative to the lowest level of ULK3 expression detected in the heart. ULK3 mRNA was detected in all tissues with highest expression in fetal brain. Postnatal tissues showed high level of ULK3 mRNA were liver, kidney and brain.

D. Quantitative RT-PCR analysis of ULK3 mRNA expression in sections of human brain.

The normalized by HPRT mRNA expression data is shown relative to the expression level in the cerebral cortex. The highest expression of ULK3 was detected in hippocampus, the lowest in spinal cord. Optic nerve, olfactory bulb and cerebellum showed increased level of ULK3 expression.

FIG. 2 depicts induction of the transcriptional activity of endogenous and overexpressed GLI1 and GLI2 by ULK3 depending on its functional kinase domain.

A. ULK3 enhances GLI1- and GLI2-dependent luciferase activity in Shh-L cells.

In Shh-L cells ULK3 is able to induce the luciferase activity from GLI-dependent promoter either alone or in cotransfection with GLI1 or GLI2. Overexpression of STK36 with or without of GLI1 and GLI2 does not result in significant induction of luciferase activity. However, ULK1 alone was able to activate luciferase activity; but it had no effect on overexpressed GLI1 and GLI2 indicating that ULK1 may influence the pathway undirectly bypassing GLI proteins.

B. ULK3 kinase acitivity is required for the regulation of GLI protein transcriptional activity.

Wt ULK3 was able to activate endogenous and overexpressed GLI1 and GLI2 dependent transcription in the presence or absence of biologically active SHH. Mutant ULK3(K44R) demonstrated residual activity in the case of activated SHH pathway and in the presence of GLI2 but not GLI1, whereas ULK3(K139R) mutant was completely inactive.

FIG. 3 depicts autophosphorylation of ULK3 and phosphorylation of GLI proteins in vitro.

A. Autophosphorylation of ULK3.

Wt and mutant ULK3 proteins were expressed in HEK293 cells and immunoprecipitated using M2-a-FLAG affinity gel (Sigma) Immunocomplexes were detected with WB using M2-a-FLAG antibody (Sigma) and subjected to in vitro kinase assay. ULK3 strongly phosphorylated itself. Mutation in Lysine 44 partly affected the autophosphorylation activity. ULK3 (K139R) lacked the autophosphorylation activity.

B. ULK3 phosphorylates GLI proteins.

FLAG-tagged GLI proteins were expressed in HEK293 cells, immunoprecipitated using M2-a-FLAG affinity gel (Sigma) and confirmed with WB using M2-a-FLAG antibody (Sigma). Aliquots of the immunoprecipitated proteins were mixed together as indicated and subjected to in vitro kinase assay. ULK3 phosphorylated strongly GLI2 and weakly GLI1 and GLI3.

C. Multiple sites in GLI1 are phosphorylated by ULK3 in vitro.

His-tagged GLI1 fragments were expressed in E. coli, purified, and detected using a-His antibody (Novagen). Purified GLI1 fragments were mixed with ULK3-M2 or vector-M2 immune complexes and in vitro kinase assay was performed. Phosphorylation of GLI1 by ULK3 was detected using constructs harboring GLI1 amino acids 1-426 and 754-1106.

FIG. 4 depicts nuclear localization of GLI1 promoted by ULK3.

A. Nuclear translocation of GLI1 induced by ULK3 is its kinase activity-dependent. NIH3T3 were cotransfected with GFP-tagged GLI1 and FLAG-tagged ULK3 constructs or empty vector. The cells were fixed 48 h after transfection. Immunostaining was performed using M2-anti-FLAG primary antibody (Sigma) and AlexaFluor-568 (Invitrogen) secondary antibody mixed with Hoechst. The cells were captured using Olympus SZ40 stereo microscope and an Olympus Camedia C-5050 digital camera. In each transfection GLI1GFP localization was estimated in approximately 75-200 FLAG-positive cells (n—nuclear localization of GLI1GFP, c—cytoplasmatic localization of GLI1GFP), and the average values from three independent experiments were calculated. Cotransfected with the empty vector, GLI1 shuttled within the cell. Under influence of wt ULK3 major part of GLI1 translocated to the nucleus. The kinase-defficient mutant ULK3(K139R) failed to change the GLI1 localization.

B. Nuclear translocation of GLI1 under influence of ULK3 was confirmed by WB.

Cos1 cells cotransfected with FLAG-tagged GLI1 and the constructs indicated. Western Blot analysis of whole cell extracts (WCE) and nuclear extracts (NE) was performed using M2-anti-FLAG antibody (Sigma) and anti-Lamin A/C (Upstate). Amount of GLI1 was comparatively equal in all WCEs. But in nuclear extract of cells cotransfected with GLI1 and ULK3 amount of GLI1 was higher than in other NEs.

FIG. 5 depicts induction of endogenous Gill expression by ULK3.

A. Quantitative RT-PCR analysis of GLI1 mRNA expression level in HEK293 cells induced by SHHC24II or transfected with ULK3.

HEK293 cells were transfected by ULK3 or respective empty vector. Afterwards the cells transfected with empty vector were induced by SHHC24II. Cells were incubated 24 h and 72 h. GLI1 mRNA expression data normalized by HPRT is shown relative to the expression level in cells transfected with the vector. During 72 h of incubation the overexpressed ULK3 induced the expression of GLI1.

B. Wt ULK3 but not a kinase-deficient mutant ULK3(K139R) induces the expression of endogenous Gli1 in HEK293 cells.

Normalized GLI1 mRNA expression data is shown relative to the expression level in the cells transfected with vector. ULK3 induced the endogenous GLI1 expression level to be approximately 6 times higher than the empty vector. ULK3(K139R) failed to activate the expression of GLI1.

### DETAILED DESCRIPTION OF THE INVENTION

Abbreviations used in this disclosure: Ci, Cubitus interruptus; CK1, Casein Kinase 1; FCS, fetal-calf serum; Fu, Fused; GSK3, Glycogen Synthase Kinase 3; IP, immunoprecipitation; KB, kinase buffer; NE, nuclear extract; PKA, Protein Kinase A; PEI, Polyethylenimine; PIC, protease inhibitor cocktail; Shh, Sonic Hedgehog; STK36, Serine/Threonine Kinase 36; qRT-PCR, quantitative real-time PCR; SWM, Stain Wash Medium; ULK, unc-51-like kinase; WCE, whole cell extract.

In this disclosure we show the cloning of human serine/threonine kinase ULK3 that has been annotated as belonging to unc-51-like family of serine/threonine kinases, but shares similarity with STK36 and dFu proteins. We show that ULK3 is an autophosphorylated kinase. In cultured cells ULK3 is able to enhance endogenous and overexpressed GLI1 and GLI2 transcriptional activity and to induce nuclear translocation of GLI1. We show that ULK3 phosphorylates GLI proteins in vitro, and GLI1 has at least two phosphorylation sites situated in N- and C-terminus of the protein. We have found that, in contrast to STK36, the kinase-deficient mutants of ULK3 are inactive indicating that functional kinase domain of ULK3 is required for the regulation of GLI protein activity. Also we show that ULK3 expression is higher in fetal brain and in a number of postnatal tissues where Shh signaling is known to be active. Our data suggests that ULK3 is involved in Shh pathway as a positive regulator of Gli proteins.

In this disclosure we identify human ULK3 as a serine/threonine kinase regulating positively Shh pathway in mammalian cells.

It has been shown previously that protein kinase dFu is absolutely required for the activation of Hh pathway in Drosophila. dFu works in a concert with Cos2, dSmo and dSufu, forming a microtubule-binding complex that controls activity of Ci transcription factor [18-20, 22, 23]. In spite of overall Hh pathway conservation between vertebrates and invertebrates, the function of those proteins is not fully conserved in mammals. In Drosophila Cos2 plays a central role in regulating the activity of Ci [40, 41], but mammalian orthologues of Cos2, Kif7 and Kif27, have no effect on Shh signaling [4]. The cytoplasmic C-terminus of dSmo, that is extremely important for Hh signaling in Drosophila, is not conserved in mammalian Smo, and, moreover, mouse Smo C-terminus is not required for Shh signal transduction [4, 22]. In Drosophila Sufu gene function is dispensable for Hh signaling and Sufu protein has only a slight negative effect on Ci, whereas mouse Sufu−/− mutants are not viable and inhibiting effect of mammalian Sufu on Gli proteins is very influential [42, 43]. Taking into account the divergences of Hh signaling in Drosophila and mammals on molecular level, it is not surprising that the role of mammalian Fu in Hh pathway also differs from that in Drosophila.

Mammalian orthologue of dFu, STK36, was identified based on sequence homology [26]. While STK36 is able to positively regulate Gli proteins in cultured cells, it is dispensible for Shh signaling during mouse development [30, 31]. Accordingly, additional kinases may be involved in the mammalian Hh pathway. This disclosure shows that ULK3 participates in Gli regulation.

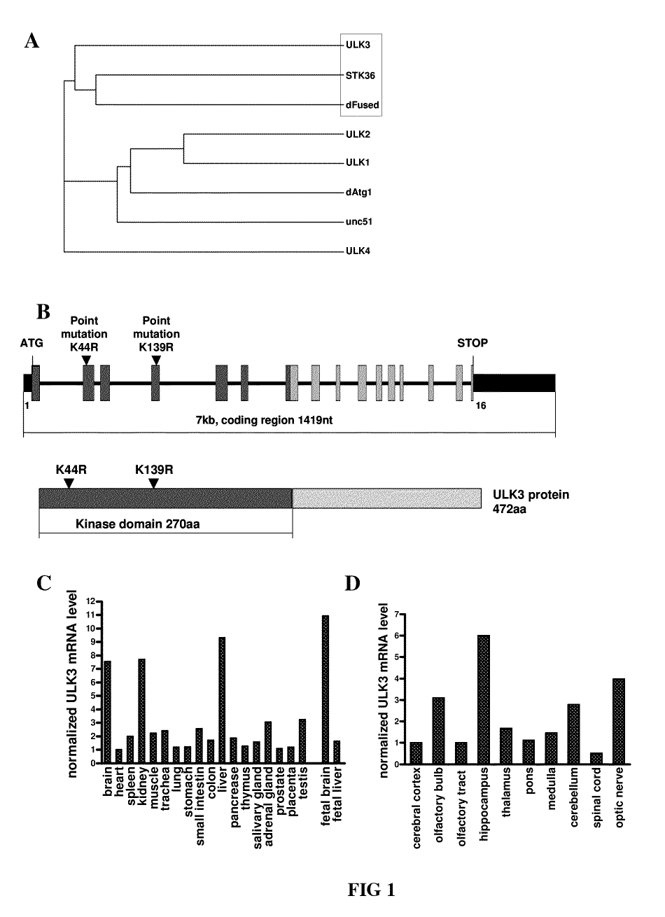

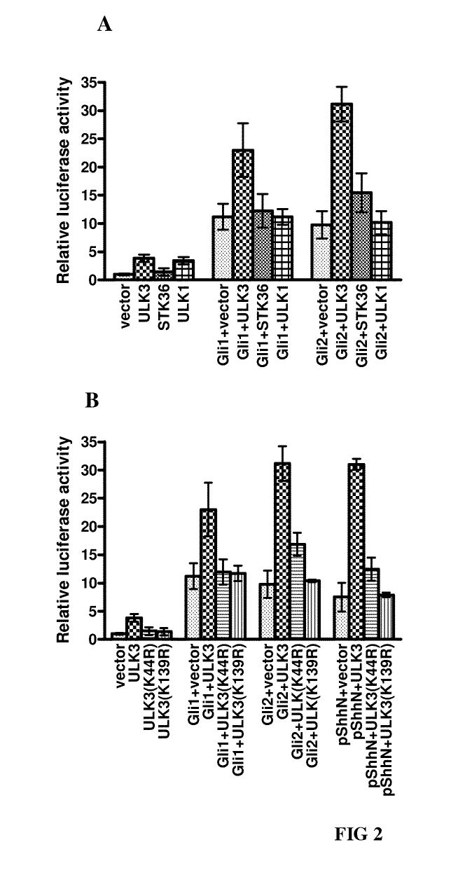

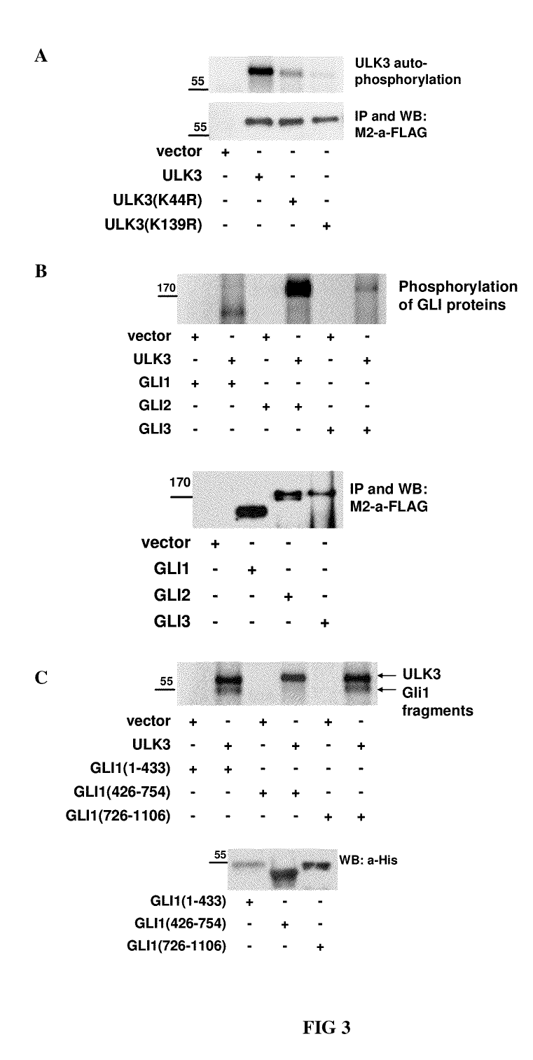

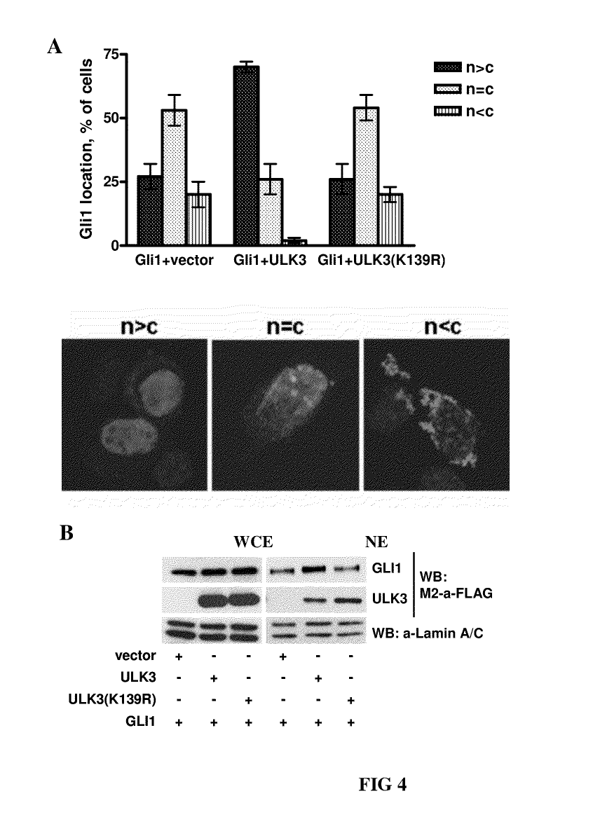

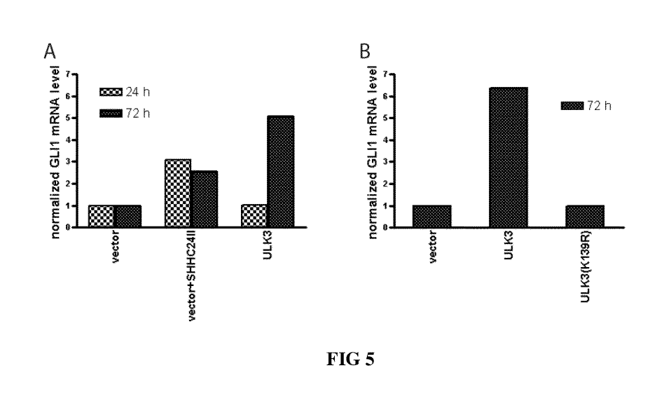

Previously, ULK3 has been annotated as belonging to the family of unc-51-like kinases that comprises also ULK1, ULK2 and ULK4 proteins. This disclosure provides however, that ULK3 diverges from other members of the family. The bioinformatic analysis suggests that ULK3 is the closest homologue of STK36 in humans (FIG. 1A). We cloned human ULK3 full-length cDNA and made two kinase inactive forms of ULK3 by introducing point mutations into the catalytic domain of the protein (FIG. 1B).

Analysis of ULK3 expression pattern reveals that ULK3 is widely expressed and its expression is higher in a number of tissues where Shh signaling is known to be active, such as postnatal brain, hippocampus, olfactory bulb, cerebellum, optic nerve, liver and fetal brain (FIGS. 1C and 1D). In the same parts of brain expression level of GLI1 and PTCH1 mRNA was also elevated (data not shown).

The role of Shh signaling in brain development is very extensive (reviewed in [44]). There is no direct evidence for Shh role in liver development, and in healthy adult liver Shh activity is low [45]. But it has been shown that cells, involved in adult liver repair, are capable of producing and responding to Hh ligands [46].

In central nervous system Shh signaling has been shown to contribute to neurogenesis that is going on in adult brain. Hippocampus is known as one of the zones where neurogenesis occurs, and Shh signaling has been shown to be implicated in control of stem cells behavior in adult hippocampus [47]. In postnatal cerebellum Shh, expressed Purkinje cells, regulates proliferation of the granule cells—the process required for cerebellar foliation (reviewed in [44]). Also Shh signaling has been shown to be active in rodent postnatal optic nerve [48]. The role of Shh signaling in olfactory bulb has not been documented yet. However, it has been shown that stem cells of subventricular zone of forebrain proliferate under the control of Shh [49]. Those cells migrate into the olfactory bulb, where they differentiate into functional interneurons [44]. The instant disclosure shows that Shh-responsive tissues express higher levels of ULK3 mRNA and accordingly that provides indirect evidence that ULK3 is involved in Shh pathway in vivo.

This disclosure shows that ULK3 (SEQ ID NO: 1) possesses kinase activity and is able to phosphorylate itself in vitro (FIG. 3A). It has been previously shown that ULK1 and ULK2 are, also autophosphorylated kinases in vitro [38, 39]. Mouse ULK1 and ULK2 are autophosphorylated in the conserved central proline/serine rich domain of the protein. However, ULK3 lacks such domain. Instead, according to bioinformatic analysis, ULK3 protein harbors a central domain contained within microtubule interacting and trafficking molecules (MIT). Besides that, the sequence mapped in dFu as Sufu-interacting domain (residues 306-436, [20]) is conserved to a certain extent in ULK3. However, C-terminal sequences responsible in dFu for interacting with Cos2 and carboxyl terminus of Smo are absent in ULK3.

This disclosure also provides that ULK3 is able to phosphorylate all three mammalian GLI proteins in vitro (FIG. 3B). Although a number of serine/threonine kinases has been proposed to be involved in modulating Hh pathway both in Drosophila and in vertebrates, only PKA has been shown to phosphorylate directly all Gli proteins, and CK1 and GSK3 have been shown to phosphorylate Gli2 and Gli3 following primary phosphorylation by PKA [36, 50-53]. PKA phosphorylates Gli1 in residues Thr-374 and Ser-640 [36]. Gli2 and Gli3 are phosphorylated by PKA, CK1, and GSK3 in a C-terminal cluster between amino acids 784-855 that corresponds to Gli1 residues 590-658 [51, 53]. According to this disclosure ULK3 phosphorylates GUI (SEQ ID NO: 2) in both N-terminus (residues 1-426) and C-terminus (residues 754-1126), but fragment of GLI1 between residues 426-754 is not phosphorylated by ULK3 (FIG. 3C).

Phosphorylation by PKA, CK1 and GSK3 has been described to elicit mainly negative effects on Gli proteins (however, see [14]). Our analysis of Shh pathway activation in cell culture identifies ULK3 as a positive regulator of the pathway. Cotransfection of ULK3 together with GLI1 or GLI2 in Shh-L2 cells shows that ULK3 is able to potentiate the transcriptional activator function of both of them (FIG. 2A).

Also, we have found that ULK3, comparing with its closest homologues ULK1 and STK36, has the strongest effect on GLI-dependent luciferase reporter activity. It has been previously reported that STK36 is a positive regulator of SHH pathway that acts independently on its functional kinase domain. STK36 is able to induce nuclear translocation of Gli1 [26]. Besides that, STK36 enhances Gill transcriptional activity in NIH3T3C2 and SW480 cells [29] and Gli2 transcriptional activity in C3H/10T and HEK293 cells [26, 27]. Here, we show that STK36 fails to induce GLI1 but induces GLI2 transcriptional activity in Shh-L2 cells. However, STK36 possesses significantly weaker coactivator potential than ULK3, probably due to lack of kinase activity. We show that kinase activity of ULK3 is required for activation of Shh pathway in cell culture (FIG. 2B) and to induce nuclear accumulation of GLI1 (FIG. 4). Together with the data showing that ULK3 is able to phosphorylate all GLI proteins in vitro, we suggest that ULK3 is pan-GLI activating kinase in mammalian cells.

The invention is now described by examples that are meant to be descriptive and by no means limiting the various embodiments of the present invention.

### EXAMPLE 1

### Materials and Methods Used throughout this Disclosure

Expression Constructs

ULK3 cDNA was amplified using primers pair sense 5′-AATGGCGGGGCCCGGCTG-3′ (SEQ ID NO:3) and anti-sense 5′-TCTGCTCCAGATGGCTCACA-3′ (SEQ ID NO:4) from human testis cDNA sample using Expand Long Template PCR System Kit (Roche Applied Science, Bazel, Switzerland) according to manufacturer's instructions. Obtained PCR product was purified from agarose gel using QIAquick Gel Extraction Kit (Qiagen, Valencia Calif., USA) and cloned to pTZ57R/T vector using InsTAclone™ PCR Cloning Kit (Fermentas, Vilnius, Lithuania).

ULK3 cDNA (SEQ ID NO:5) was verified by sequencing and subcloned to mammalian expression vectors. ULK3pcDNA3.1 construct was generated by cloning of ULK3 cDNA into KpnI and BamHI linearized pcDNA3.1 vector (Invitrogen, Carlsbad Calif., USA). ULK3FLAG construct was produced by cloning of ULK3 cDNA into EcoRI and HinduI sites of pFLAG-CMV-4 vector (Sigma-Aldrich). ULK3(K44R) (SEQ ID NO:6) with Lysine residue at position 44 mutated to Arginine and ULK3(K139R) (SEQ ID NO:7) harboring the same mutation at position 139 were generated from ULK3FLAG construct by Quickchange site directed PCR mutagenesis procedure (Stratagene, La Jolla Calif., USA) using Expand Long Template PCR System Kit (Roche Applied Science) and oligos carrying the appropriate point mutations. The obtained constructs were verified by DNA sequencing.

N-terminally tagged GLI1GFP and GLI1FLAG constructs have been described in [32]. GLI2FLAG and GLI3FLAG constructs were constructed in Tallinn University of Technology. GLI2FLAG was generated by subcloning GLI2 cDNA from GLI2pcDNA3 described in [33] into HindIII and XbaI sites of pFLAG-CMV-4 vector. GLI3pcDNA3.1 construct (described in [34]) was used for generation of GLI3FLAG by subcloning of GLI3 cDNA to pFLAG-CMV-4 vector. STK36pcDNA3.1 has been described in [27].

Expression pattern of ULK3

cDNA panels of 20 human tissues and 10 human brain parts were used in this experiment. Levels of ULK3 mRNA and mRNA of housekeeping gene HPRT used for normalization were detected in triplicates by quantitative Real-Time PCR using qPCR Core kit for SYBR Green (Eurogentec, Oslo, Norway) with Lightcycler 2.0 (Roche Applied Science) according to the manufactures' instructions. Data was analyzed with Lightcycler 4.05 software (Roche Applied Science). The following primers were used for the assay:

(SEQ ID NO: 8)

| | |
|---|---|
| ULK3 sense | 5′-AAGGAGCAGGTCAAGATGAG-3′ |

(SEQ ID NO: 9)

| | |
|---|---|
| ULK3 antisense | 5′-GTGCAAGAGCTACGAACAGA-3′ |

(SEQ ID NO: 10)

| | |
|---|---|
| HPRT sense | 5′-GATGATGAACCAGGTTATGAC-3′ |

(SEQ ID NO: 11)

| | |
|---|---|
| HPRT antisense | 5′-GTCCTTTTCACCAGCAAGCTTG-3′ |

Cell Culture

HEK293 (human embryonic kidney-293) cells were propagated in Minimum Essential Medium (MEM) (Gibco, Invitrogen). NIH3T3 (mouse embryonic fibroblasts) and their clone Shh-Light2 cells [35] were grown in Dulbecco's modified Eagle's medium (D-MEM) contained 4.5 g/L glucose (Gibco). Cos1 cell line (primate kidney fibroblasts) was grown in DMEM contained 1 g/L glucose (Gibco). All growth media were supplemented with 10% FBS (PAA) and 100 μg/ml of penicillin/streptomycin (Invitrogen), and Shh-L2 cells growth medium was additionally supplemented with 400 μg/ml of G 418 (Sigma-Aldrich-Aldrich) and 100 μg/ml of Zeocine (Invitrogen). The cells were grown at 37° C. and 5% CO₂. Approximately 24 h prior to transfection the cells were plated to appropriate growth dishes.

Overexpression Studies

Cells were transfected by the expression constructs or respective empty vectors using Polyethylenimine transfection agent (PEI) (Inbio) and as described in [28]. We used 0.25 μg of cDNA per 1 cm² of plate surface area for transfections. After 3 h of transfection HEK293, NIH3T3 and Cos1 cells were propagated in the normal growth medium for 48 h. Prior the further analysis the cells were washed twice with PBS. Post-transfectional Shh-L2 cells were grown in the normal growth medium for 24 h and for additional 24 h in the Light medium containing 0.25% FBS, washed once with PBS, lysed in Passive Lysis Buffer (Promega, Madison Wis., USA) (70 ul per well of 24-well plate format) and subjected to luciferase assay.

Luciferase Assay

Luciferase assay was performed as previously described [28,36]. Briefly, firefly luciferase activity of 12 μl of the Shh-L2 cells lysate was measured using Luciferase Assay Kit (Promega) and galactosidase activity was quantified using Galacto-LightPlus kit (Tropix, Bedford Mass., USA) according to the manufacturer's instructions. Chemiluminicence was measured using Ascent FL Fluoroscan (Thermo Electron Corporation, Waltham Mass., USA) according to the manufacturer's instructions.

Purification of GLI1 Fragments

Three overlapping human GLI1 domains corresponding to amino acids 1-433, 426-754 and 726-1106 were cloned into pET-15b vector (Novagen) between NdeI and BamHI sites. GLI1 amino acid sequence is provided as SEQ ID NO: 2). The fragments were expressed in BL21(DE3)pLys E. coli strain overnight at 21° C. using 0.1 mM IPTG and purified using Ni-CAM™ HC resin (Sigma-Aldrich) according to the manufacturer's recommendations. The proteins were eluted by PBS and verified by Western Blot (WB).

Immunoprecipitation

HEK293 cells were transfected with 13.5 μg of FLAG-tagged constructs expressed GLI1, GLI2, GLI3, ULK3, ULK3(K44R), ULK3(K139R) or FLAG-CMV-4 empty vector on 10 cm plates. The cells were lysed with 0.5 ml of Lysis Buffer (50 mM Tris HCl, pH 7.4, 150 mM NaCl, 1 mM EDTA, 1% Triton X-100) containing protease inhibitor cocktail (PIC) (Roche Applied Science). The lysates were centrifuged for 15 min at 4° C. and 15000 g, and the supernatants were used for immunoprecipitation (IP). IP was performed using anti-FLAG-M2 affinity gel (Sigma-Aldrich) in batch format according to the manufacture's instructions. Immune complexes were washed three times with 500 μl of Kinase Buffer (KB) (50 mM HEPES, pH 7.4, 20 mM MgCl₂, 25 μM ATP) and resuspended in 12.5 μl of KB. One fifth of the immuno-complexes was subjected to WB and the rest was used for the kinase assay.

In Vitro Kinase Assay

Kinase reactions were carried out in KB in a total volume of 20 μl of in the presence of 1 μCi of [γ-³²P]ATP per reaction. We used 1 μl of the immunoprecipitated ULK3, ULK3(K44R) or ULK3(K139R) mixed with 5 μl of the GLI-M2 immunocomplexes or 5 μl of bacterially expressed and purified His-GLI1 fragments. Kinase reactions were held at 30° C. for 30 min and terminated by adding of 5 μl of 4× Laemmli Sample Buffer. Proteins were resolved by SDS-PAGE. The gel was dried at 80° C. for 2 h, and autoradiography was performed using Bio-Rad Personal Molecular Imager FX.

Immunocytochemistry

NIH3T3 cells were transfected with GLI1GFP and FLAG-tagged expression constructs for ULK3 or ULK3(K139R) in ratio 1:1 on 8-chamber slides (Falcon, BD Biosciences, San Jose Calif., USA). The cells were fixed with 4% paraformaldehyde and washed three times for 5 min with Stain Wash Medium (SWM) (0.5% BSA and 0.01% NaN₃ in PBS). Subsequent permeabilization of cells was performed at RT by 10-min incubation in. PBS supplemented with 0.5% Triton X-100. Afterwards, the cells were washed three times for 5 min with SWM, blocked for 30 min in PBS containing 5% bovine serum albumin (BSA), and incubated with mouse monoclonal M2-anti-FLAG antibody (diluted 1:1000 in SWM) for 1 h at RT with gentle agitation. After three washes of 10 min with SWM, the cells were incubated for 30 min with secondary antibody Alexa-Fluor 568 (Invitrogen) (diluted 1:500 in SWM) mixed with nuclear stain Hoechst 33342 (diluted 1:100). Cells were washed twice with SWM and mounted with Mowiol 4-88 (Sigma-Aldrich). GLI1GFP protein localization was assessed in at least 200 FLAG-positive cells under a fluorescent microscope Olympus BX61 with UPLan SApo 40× objective, and the experiment was repeated three times. Confocal images were obtained with a Zeiss LSM-510 META confocal laser-scanning microscope (Carl Zeiss MicroImaging GmbH, Germany) equipped with Plan-Apochromat 63×/1.4 oil immersion objective.

Subcellular Fractionation

Cos1 cells were transfected with FLAG-tagged expression constructs for GLI1 combined with ULK3, ULK3(K139R) or empty vector in ratio 1:1 on 10 cm plates. Nuclear and whole cell extracts (NE and WCE, respectively) were prepared as described in [36]. All extracts were normalized for protein amounts measured using BSA kit (Thermo Scientific). A total 10 μg of protein were separated by SDS-PAGE and transferred to the PVDF membrane (Millipore, Billerica Mass., USA). The membrane was segmented according to the expected size of the detected proteins. Obtained strips were probed with the appropriate antibodies.

Antibodies and WB Procedure

WB was performed using a non-blocking technique as described in [37]. Mouse monoclonal M2-anti-FLAG antibody (Sigma-Aldrich) was used for detection of FLAG-tagged GLI1, GLI2, GLI3, ULK3, ULK3(K44R) and ULK3(K139R) by WB in dilution 1:2000. Lamin A/C was detected with mouse monoclonal anti-Lamin A/C antibody (Upstate, Billerica Mass., USA) diluted 1:1000. Mouse monoclonal anti-His antibody (Novagen, Darmstadt, Germany) diluted 1:1500 was used for detection of bacterially expressed and purified GLI1 fragements. The secondary antibody used was HRP-conjugated goat-anti-mouse Ig diluted 1:1000.

Quantitative RT-PCR Analysis of GU1 mRNA Expression Level

HEK293 cells were transfected with FLAG-tagged wt ULK3, ULK3(K139R) or respective empty vector in two replicates. After 5 h of incubation one replicate of cells transfected with the vector was induced by SHHC24II. The cells were incubated 24 h and 72 h, washed with PBS. Total RNA was isolated and treated with DNaseI using RNAqueous kit (Ambion) according to manufacture's instructions. cDNA of 2 μg total RNA was synthesised using SuperScript III kit (Ambion). Levels of GLI1 mRNA and mRNA of housekeeping gene HPRT used for normalization were detected in triplicates by quantitative Real-Time PCR. The following GLI1 primers were used for the assay:

| (SEQ ID NO: 12) | |
|---|---|
| GLI1 sense | 5′-CCTTCAGCAATGCCAGTGA-3′ |
| (SEQ ID NO: 13) | |
| GLI1 antisense | 5′-CTAGGATCTGTATAGCGTTT-3′ |

### EXAMPLE 2

### ULK3 is the Closest Homologue of STK36

In order to find out the kinases homologous to human STK36 (GenBank accession number NP056505.2), sequence of its kinase domain was subjected to comparative homology analysis against human proteins databank using BLASTp algorithm and BLOSUM62 matrix (NCBI). Our analysis revealed that ULK3 (accession number NP001092906) was a protein sharing the highest homology with STK36 (38% of identity in the kinase domain). Using the sequence of kinase domain of dFu (accession number NP477499.1) as a query, we found that ULK3 was the second homolog of dFused protein after STK36 in human. However, the identity between ULK3 and dFused kinase domains was lower comparing to that of STK36 (37% vs 51%).

On the other hand ULK3 (SEQ ID NO: 1) was found to be homologous to ULK1 and ULK2 proteins that belong to unc51 subfamily (39% and 37% of identity in the kinase domain, respectively [38, 39]). The sequences of the members of the unc51 subfamily were obtained from NCBI database (accession numbers NP_{—}507869 for unc51 (C. elegance), NP648601 for Atg1 (D. melanogaster), NP003556 for ULK1, NP055498 for ULK2, NP060356 for ULK4). The proteins of fused and unc51 subfamilies of serine/threonine kinases were subjected to multiple sequence alignment and homology analysis using GONNET 250 matrix and ClustalW program (EBI, EMBL). The homology tree was built using the calculated distances between the aligned proteins. Pairwise aligment of the proteins showed that, ULK3 shared higher homology to Fu subfamily of serine/threonine kinases than to unc-51-like kinases (FIG. 1A). Taken together, the bioinformatic analysis suggests that ULK3 is a homologue of STK36.

### EXAMPLE 3

### Cloning of ULK3 and Generation of Kinase-Deficient Mutants

The comparative analysis of ULK3 nucleotide sequence against human GenBank was performed using UCSC Genome Browser. The analysis revealed ULK3 gene is situated in chromosome 15 and contains 16 exons with translation initiation codon in the first exon and STOP codon in the exon 16 (FIG. 1B).

ULK3 cDNA was amplified by RT-PCR from adult human testis cDNA using the pair of primers complementary to the predicted coding part of ULK3. The obtained 1419nt long cDNA (SEQ ID NO: 5) corresponded to ULK3 mRNA sequence (GenBank accession number NM001099436.1) It encodes a polypeptide of 472 amino acids (SEQ ID NO: 1) with calculated molecular weight of 53 kDa and contains N-terminal serine/threonine kinase domain (amino acids 14-270). (SEQ ID NO: 14)

In order to produce hypothetically kinase-inactive variants of ULK3, we mutated the highly conserved lysines in positions 44 and 139 to arginines (ULK3(K44R)(SEQ ID NO:6) and ULK3(K139R)(SEQ ID NO:7)). Lysin residue in position 44 is situated in the ATP binding pocket, and lysine in position 139 is situated in the substrate binding pocket and catalytic loop regions. The substitutions were made by site directed PCR mutagenesis procedure using the oligos carrying the appropriate mutations.

### EXAMPLE 4

### ULK3 mRNA is Widely Expressed in Humans with the Highest Expression in Fetal Brain

The level of ULK3 mRNA expression was analyzed in 20 human tissues including fetal brain and fetal liver using Quantitative Real-Time PCR. ULK3 mRNA was detected in all tissues analyzed and the data is shown relative to the level in the heart as a tissue with the lowest level of ULK3 mRNA expression (FIG. 1C). The highest expression of ULK3 was detected in fetal brain. Post-natal tissues showing high level of ULK3 expression were brain, liver and kidney; moderate amount of ULK3 mRNA expression was detected in testis and adrenal gland. Heart, lung, stomach, thymus, prostate and placenta showed low level of ULK3 expression.

As the expression of ULK3 in the brain was higher than in most other tissues, we were interested to know if particular regions of adult human brain are responsible for high level of ULK3 expression. We analyzed 10 brain regions and found the highest level of ULK3 expression in hippocampus (FIG. 1D). The data is shown relative to the expression level in the cerebral cortex. Higher levels of ULK3 mRNA were detected in cerebellum, olfactory bulb and optic nerve. The lowest level of ULK3 expression was detected in spinal cord.

Analysis of ULK3 expression suggests that ULK3 may contribute to brain development as well as play a role in adult organism.

### EXAMPLE 5

### ULK3 Kinase Activity is Required to Enhance GLI-Dependent Luciferase Activity

To show that ULK3 plays a role in Shh pathway we examined if ULK3 and its closest homologues ULK1 and STK36 are able to activate GLI-dependent luciferase activity in Shh-LIGHT2 cells (Shh-L2) cells. We cotransfected the constructs together with GLI-expressing plasmids (or respective empty vector) into Shh-L2 cells and assessed their effect on GLI-dependent firefly luciferase activity. The obtained data was normalized with β-galactosidase values. The experiment was repeated 4 times, and results of the representative experiment are shown in FIG. 2A. Among the kinases tested, ULK3 surprisingly demonstrated the strongest effect on GLI-dependent luciferase reporter activity either alone or cotransfected with GLI1 and GLI2. It was able to stimulate the luciferase activity 3.8 times and enhanced the transcriptional activity of over expressed GLI1 and GLI2 approximately 2 and 3.2 times, respectively. The potency of STK36 in the assay was much lower. STK36 alone failed to activate the luciferase activity and exerted no effect on GLI1; however it enhanced GLI2 activity 1.6 times. It is noticeable that ULK1 alone was able to activate luciferase activity 3.4 times; however it had no effect on overexpressed GLI1 and GLI2 indicating that ULK1 may influence the Shh pathway bypassing GLI proteins.

In order to examine if the kinase activity of ULK3 is required for the luciferase reporter activation we tested the supposed kinase-deficient variants of ULK3, ULK3(K44R) and ULK3(K139R) in the same assay. In contrast to ULK3, neither of the mutants had an effect on GLI1 transcriptional activity. However, mutant ULK3(K44R), if coexpressed with GLI2, demonstrated some residual activity, whereas ULK3(K139R) was completely inactive (FIG. 2B). We also tested the effect of ULK3 and the mutants on the luciferase reporter when the SHH pathway was activated by cotransfection with pShhN (plasmid expressing biologically active part of SHH). ULK3 significantly affected the activated pathway inducing the luciferase activity approximately 4.1 times. Neither of the mutants could activate the Gli-luciferase reporter as efficiently as wild-type ULK3, although ULK3(K44R) had a residual positive effect on the activated pathway inducing luciferase activity 1.7 times.

These data indicate that in contrast to STK36 and ULK1, ULK3 is able to positively regulate the GLI transcriptional activity and its kinase activity is required for that.

### EXAMPLE 6

### ULK3 has Kinase Activity and Phosphorylates GLI Proteins In Vitro

To test if ULK3 has an autophosphorylation activity, we expressed FLAG-tagged wild-type ULK3 and mutants ULK3(K44R) and ULK3(K139R) in HEK293 cells, immunopurified and subjected to in vitro kinase assay in the presence of γ-P³²-ATP. ULK3 demonstrated strong autophosphorylation activity, ULK3(K44R) showed reduced efficiency in autophosphorylation, and ULK3(K139R) autophosphorylation activity was almost completely absent (FIG. 3A). This data proves that ULK3 is an autophosphorylated kinase. Its activity in vitro is altered by the mutations in lysines 44 and 139—partly inhibited by mutation in lysine 44 or entirely destroyed by mutation in position 139.

We also assayed whether ULK3 is able to phosphorylate GLI proteins. FLAG-tagged GLI1, GLI2 and GLI3 proteins were expressed and immunoprecipitated from HEK293 cells and used as substrates for immunopurified ULK3 in the in vitro kinase assay. ULK3 was able to phosphorylate GLI proteins but with different efficiency (FIG. 3B). The strongest phosphorylation signal was detected in the case of GLI2. The mutant ULK3(K44R) could slightly phosphorylate GLI2 (data not shown) which is consistent with the luciferase assay data. GLI1 and GLI3 were also phosphorylated by ULK3, but with significantly lower intensity as compared to GLI2. To support these findings and identify the regions in GLI1 protein that are phosphorylated by ULK3, we used bacterially expressed His-tagged fragments of GLI1 as substrate for ULK3 kinase. Two of them—GLI1(1-433) and GLI1(726-1106)—were phosphorylated by ULK3. Central part of GLI1 (amino acids 426-754) was not phosphorylated by ULK3. This data shows that ULK3 phosphorylates directly GLI proteins and GUI has at least two phosphorylation sites situated in N- and C-terminus.

### EXAMPLE 7

### ULK3 Promotes Nuclear Localization of GLI1 and Kinase Activity is Essential to the Function

Next we examined if overexpression of ULK3 kinase influences the subcellular localization of GLI1 and GLI2 proteins. As ULK3(K44R) demonstrated the residual activity both in the luciferase and in vitro kinase assays and ULK(K139R) was completely inactive, we preferred the latter as a negative control in the immunofluorescence assay. FLAG-tagged ULK3 and ULK3(K139R) (or respective empty vector) were coexpressed with GFP-tagged GLI1 and GLI2 in NIH3T3 cells. Subcellular localization of GLI1 and GLI2 was determined, and average values were calculated from three independent experiments. ULK3 and its mutant remained almost completely cytoplasmic. GFP-tagged GLI2 localized predominantly in the nucleus and its localization was not altered by ULK3 or ULK3(K139R) (data not shown). Overexpressed GLI1GFP was detected both in cytoplasm and nucleus (FIG. 3A). The distribution of GLI1 was the following: 27% of cells showed stronger signal in the nucleus, GLI1 was cytoplasmic in 20% of cells, and in 53% of cells GLI1 was distributed uniformly within the cell. Expression the kinase-deficient mutant ULK3(K139R) did not influence GUI subcellular localization, the distribution of GLI1 between nuclei and cytosol remained unchanged. Under the influence of ULK3 localization of GLI1 was shifted. GLI1 was detected mostly in the nucleus in 70% of cells, only in 2% of cells retained GUI in the cytoplasm, and 25% of cells demonstrated the uniform distribution of GLI1.

To confirm the data from the immunofluorescent staining, we performed cell fractionation analysis. FLAG-tagged GLI1 and ULK3 or ULK3(K139R) (or the respective empty vector) were coexpressed in Shh-unresponsive Cos1 cells, and the cells were fractionated to whole cell and nuclear extracts (WCE and NE, respectively). The extracts were used in WB with anti-FLAG and anti-Lamin A/C antibodies. Three independent experiments were done with similar results, and the data of representative experiment are shown in FIG. 4B. Expression of lamin A/C was analyzed as a loading control. ULK3 and ULK3(K139R) were detected mostly in WCEs. All WCEs showed comparably equal expression of GLI1. Analysis of NEs revealed that cells cotransfected with GLI1 and ULK3 retained most of the GLI1 in the nuclei. Cells cotransfected with GLI1 and ULK3(K139R) or empty vector demonstrated equal but significantly lower amounts of GLI1 in the nuclear extracts. This data demonstrates that ULK3 alters the subcellular localization of GLI1 but not GLI2, and the kinase activity of ULK3 is needed to induce the nuclear translocation of GLI1.

### EXAMPLE 8

### Wild-Type ULK3 but not a Kinase-Deficient Mutant ULK3K139R Induces the Expression of Endogenous GM in HEK293 Cells

Next we tested if ULK3 is able to induce the endogenous GLI1 expression. HEK293 cells were transfected by ULK3 or respective empty vector. Cells transfected with empty vector were induced by 12 nM of SHHC24II and were used as a posititve control. Cells were incubated 24 h and 72 h, and total RNA was isolated. Level of GLI1 expression was measured using QRT-PCR procedure and the obtained data was normalized by expression of housekeeping gene HPRT. Level of GLI1 expression in cells transfected with empty vector was taken as 1. During 72 h over expressed ULK3 was able to induce the expression of GLI1 in HEK293 cells (FIG. 5A). However, the kinase-deficient mutant ULK3(K139R) failed to activate the expression of GUI (FIG. 5B). This data proves that overexpressed ULK3 induces the endogenous expression of GUI in kinase activity-dependent manner.

### EXAMPLE 9

### Determination of ULK3 Phosphorylation Sites in GM

As is shown in Example 6 ULK3 phosphorylates GLI1 in both N-terminus (residues 1-426) and C-terminus (residues 754-1106). However, the exact sites of phosphorylation have to be determined using mass spectrometry analysis. We have found the phosphorylated peptide in C-terminus of GLI1: SGSYPTPSPCHENFVVGANR (SEQ ID NO: 15). This sequence corresponds to GLI1 amino acid residues 961-981 and contains 4 potential phosphorylation sites (3 serine residues and 1 threonine residue). As is evident from this disclosure, the kinase activity of ULK3 is essential factor in Shh signaling. Accordingly, identification of the phosphorylated peptide on C-terminus of GLI1 is an essential tool in regulation of the signalling pathway. Specifically, it can be used to design a high affinity inhibitor for the ULK3 kinase that is a drug candidate to enter further development.

### EXAMPLE 10

### An Inhibitor Molecule for Serine/Threonine Kinase ULK3

As is shown in the above examples, ULK3 is a serine/threonine kinase positively regulating the mammalian Hh signaling pathway. The importance of Hh signaling pathway is well known in the art and various human conditions are known to be affected by the activation/inactivation of the pathway. Accordingly our novel finding provides novel means to control the pathway and provide a target for various pharmaceutical approaches. An inhibitor of the ULK3 kinase would be a desired molecule to control the Hh signaling pathway.

The inhibitor of protein kinase ULK3 may be designed as a competitor molecule directed to bind the kinase active site. The binding affinity of this molecule towards ULK3 may be higher or comparable to the affinities of the ULK3 substrates.

The inhibitor molecule may be designed to bind the ATP binding site, the protein/peptide substrate binding site, or it may be a bifunctional inhibitor binding simultaneously both of these sites.

The inhibitor molecule can structurally mimic the adenosine moiety of ATP, the ribose moiety or phosphate moiety of ATP, or it can be any molecule exhibiting affinity and specificity to the ATP binding site of ULK3.

The inhibitor competing with the protein/peptide substrate may contain a pseudosubstrate sequence based on a phosphorylation site sequence of GLI, the physiological substrate of ULK3, or some other substrate. In the case of a pseudosubstrate, the phosphoacceptor serine or threonine could be mutated to alanine or some other amino acid. As one of the phosphorylation sites in GLI1 has been identified to be in SEQ ID NO:15 in example 9 above, the inhibitor molecule could be a pseudosubstrate based on this sequence. Other inhibitor molecules can be designed once further phosphorylation sites have been identified.

The bifunctional inhibitors could mimic the interactions of both ATP and protein/peptide substrates with the ULK3 active site. The inhibitor may also be a peptidomimetic compound or any other molecule that exhibits strong binding affinity to the ATP binding site or the protein/peptide substrate-binding site of ULK3.

In the above-described cases, the inhibitor may use similar or different interactions compared to the substrates when bound to ULK3 as far it occupies the substrate binding sites in such a way that the binding of physiological substrates is hindered.

Alternatively, the inhibitor may be designed to bind the hydrophilic region in the C-terminal non-kinase domain of ULK3. This region bears sequence and functional homology to the Drosophila analog of the kinase and has been shown to be a binding site for the protein interaction partners of the kinase. Such inhibitor may also be a multifunctional inhibitor containing both the active site binding moiety and the hydrophilic region binding moiety covalently connected to each other by a linker. For example, this linker may be a synthetic aliphatic linker, or a peptide linker.

In case the inhibitor is cell permeable, for example, a small-molecule ATP-competitive inhibitor, it can be used as a drug molecule that interferes with the ULK3 regulatory function in the SHH pathway. The inhibitor could be designed to contain a specific cell-penetrating agent, for example, a cell penetrating peptide sequence as part of the inhibitor molecule.

### REFERENCES

1. Hooper, J. E. and Scott, M. P. (2005) Communicating with Hedgehogs. Nat Rev Mol Cell Biol 6, 306-317

2. McMahon, A. P., Ingham, P. W. and Tabin, C. J. (2003) Developmental roles and clinical significance of hedgehog signaling. Curr Top Dev Biol 53, 1-114

3. Osterlund, T. and Kogerman, P. (2006) Hedgehog signalling: how to get from Smo to Ci and Gli. Trends Cell Biol 16, 176-180

4. Varjosalo, M., Li, S. P. and Taipale, J. (2006) Divergence of hedgehog signal transduction mechanism between Drosophila and mammals. Dev Cell 10, 177-186

5. Nguyen, V., Chokas, A. L., Stecca, B. and Ruiz i Altaba, A. (2005) Cooperative requirement of the Gli proteins in neurogenesis. Development 132, 3267-3279

6. Lum, L. and Beachy, P. A. (2004) The Hedgehog response network: sensors, switches, and routers. Science 304, 1755-1759

7. Varjosalo, M., Bjorklund, M., Cheng, F., Syvanen, H., Kivioja, T., Kilpinen, S., Sun, Z., Kallioniemi, O., Stunnenberg, H. G., He, W. W., Ojala, P. and Taipale, J. (2008) Application of active and kinase-deficient kinome collection for identification of kinases regulating hedgehog signaling. Cell 133, 537-548

8. Preat, T., Therond, P., Lamour-Isnard, C., Limbourg-Bouchon, B., Tricoire, H., Erk, I., Mariol, M. C. and Busson, D. (1990) A putative serine/threonine protein kinase encoded by the segment-polarity fused gene of Drosophila. Nature 347, 87-89

9. Riobo, N. A., Haines, G. M. and Emerson, C. P., Jr. (2006) Protein kinase C-delta and mitogen-activated protein/extracellular signal-regulated kinase-1 control GLI activation in hedgehog signaling. Cancer Res 66, 839-845

10. Riobo, N. A., Lu, K., Ai, X., Haines, G. M. and Emerson, C. P., Jr. (2006) Phosphoinositide 3-kinase and Akt are essential for Sonic Hedgehog signaling. Proc Natl Acad Sci USA 103, 4505-4510

11. Chen, Y., Gallaher, N., Goodman, R. H. and Smolik, S. M. (1998) Protein kinase A directly regulates the activity and proteolysis of cubitus interruptus. Proc Natl Acad Sci USA 95, 2349-2354

12. Alves, G., Limbourg-Bouchon, B., Tricoire, H., Brissard-Zahraoui, J., Lamour-Isnard, C. and Busson, D. (1998) Modulation of Hedgehog target gene expression by the Fused serine-threonine kinase in wing imaginal discs. Mech Dev 78, 17-31

13. Jia, J., Amami, K., Wang, G., Tang, J., Wang, B. and Jiang, J. (2002) Shaggy/GSK3 antagonizes Hedgehog signalling by regulating Cubitus interruptus. Nature 416, 548-552

14. Kaesler, S., Luscher, B. and Ruther, U. (2000) Transcriptional activity of GLI1 is negatively regulated by protein kinase A. Biol Chem 381, 545-551

15. Mao, J., Maye, P., Kogerman, P., Tejedor, F. J., Toftgard, R., Xie, W., Wu, G. and Wu, D. (2002) Regulation of Gli1 transcriptional activity in the nucleus by Dyrkl. J Biol Chem 277, 35156-35161

16. Preat, T., Therond, P., Limbourg-Bouchon, B., Pham, A., Tricoire, H., Busson, D. and Lamour-Isnard, C. (1993) Segmental polarity in Drosophila melanogaster: genetic dissection of fused in a Suppressor of fused background reveals interaction with costal-2. Genetics 135, 1047-1062

17. Therond, P., Alves, G., Limbourg-Bouchon, B., Tricoire, H., Guillemet, E., Brissard-Zahraoui, J., Lamour-Isnard, C. and Busson, D. (1996) Functional domains of fused, a serine-threonine kinase required for signaling in Drosophila. Genetics 142, 1181-1198

18. Ascano, M., Jr., Nybakken, K. E., Sosinski, J., Stegman, M. A. and Robbins, D. J. (2002) The carboxyl-terminal domain of the protein kinase fused can function as a dominant inhibitor of hedgehog signaling. Mol Cell Biol 22, 1555-1566

19. Malpel, S., Claret, S., Sanial, M., Brigui, A., Piolot, T., Daviet, L., Martin-Lanneree, S. and Plessis, A. (2007) The last 59 amino acids of Smoothened cytoplasmic tail directly bind the protein kinase Fused and negatively regulate the Hedgehog pathway. Dev Biol 303, 121-133

20. Monnier, V., Dussillol, F., Alves, G., Lamour-Isnard, C. and Plessis, A. (1998) Suppressor of fused links fused and Cubitus interruptus on the hedgehog signalling pathway. Curr Biol 8, 583-586

21. Claret, S., Sanial, M. and Plessis, A. (2007) Evidence for a novel feedback loop in the Hedgehog pathway involving Smoothened and Fused. Curr Biol 17, 1326-1333

22. Lum, L., Zhang, C., Oh, S., Mann, R. K., von Kessler, D. P., Taipale, J., Weis-Garcia, F., Gong, R., Wang, B. and Beachy, P. A. (2003) Hedgehog signal transduction via Smoothened association with a cytoplasmic complex scaffolded by the atypical kinesin, Costal-2. Mol Cell 12, 1261-1274

23. Ruel, L., Rodriguez, R., Gallet, A., Lavenant-Staccini, L. and Therond, P. P. (2003) Stability and association of Smoothened, Costal2 and Fused with Cubitus interruptus are regulated by Hedgehog. Nat Cell Biol 5, 907-913

24. Nybakken, K. E., Turck, C. W., Robbins, D. J. and Bishop, J. M. (2002) Hedgehog-stimulated phosphorylation of the kinesin-related protein Costal2 is mediated by the serine/threonine kinase fused. J Biol Chem 277, 24638-24647

25. Dussillol-Godar, F., Brissard-Zahraoui, J., Limbourg-Bouchon, B., Boucher, D., Fouix, S., Lamour-Isnard, C., Plessis, A. and Busson, D. (2006) Modulation of the Suppressor of fused protein regulates the Hedgehog signaling pathway in Drosophila embryo and imaginal discs. Dev Biol 291, 53-66

26. Murone, M., Luoh, S. M., Stone, D., Li, W., Gurney, A., Armanini, M., Grey, C., Rosenthal, A. and de Sauvage, F. J. (2000) Gli regulation by the opposing activities of fused and suppressor of fused. Nat Cell Biol 2, 310-312

27. Osterlund, T., Everman, D. B., Betz, R. C., Mosca, M., Nothen, M. M., Schwartz, C. E., Zaphiropoulos, P. G. and Toftgard, R. (2004) The FU gene and its possible protein isoforms. BMC Genomics 5, 49

28. Maloveryan, A., Finta, C., Osterlund, T. and Kogerman, P. (2007) A possible role of mouse Fused (STK36) in Hedgehog signaling and Gli transcription factor regulation. J Cell Commun Signal 1, 165-173

29. Daoud, F. and Blanchet-Tournier, M. F. (2005) Expression of the human FUSED protein in Drosophila. Dev Genes Evol 215, 230-237

30. Chen, M. H., Gao, N., Kawakami, T. and Chuang, P. T. (2005) Mice deficient in the fused homolog do not exhibit phenotypes indicative of perturbed hedgehog signaling during embryonic development. Mol Cell Biol 25, 7042-7053

31. Merchant, M., Evangelista, M., Luoh, S. M., Frantz, G. D., Chalasani, S., Carano, R. A., van Hoy, M., Ramirez, J., Ogasawara, A. K., McFarland, L. M., Filvaroff, E. H., French, D. M. and de Sauvage, F. J. (2005) Loss of the serine/threonine kinase fused results in postnatal growth defects and lethality due to progressive hydrocephalus. Mol Cell Biol 25, 7054-7068

32. Kogerman, P., Grimm, T., Kogerman, L., Krause, D., Unden, A. B., Sandstedt, B., Toftgard, R. and Zaphiropoulos, P. G. (1999) Mammalian suppressor-of-fused modulates nuclear-cytoplasmic shuttling of Gli-1. Nat Cell Biol 1, 312-319

33. Speek, M., Njunkova, O., Pata, I., Valdre, E. and Kogerman, P. (2006) A potential role of alternative splicing in the regulation of the transcriptional activity of human GLI2 in gonadal tissues. BMC Mol Biol 7, 13

34. Tsanev, R., Tiigimagi, P., Michelson, P., Metsis, M., Osterlund, T. and Kogerman, P. (2009) Identification of the gene transcription repressor domain of Gli3. FEBS Lett 583, 224-228

35. Taipale, J., Chen, J. K., Cooper, M. K., Wang, B., Mann, R. K., Milenkovic, L., Scott, M. P. and Beachy, P. A. (2000) Effects of oncogenic mutations in Smoothened and Patched can be reversed by cyclopamine. Nature 406, 1005-1009

36. Sheng, T., Chi, S., Zhang, X. and Xie, J. (2006) Regulation of Gli1 localization by the cAMP/protein kinase A signaling axis through a site near the nuclear localization signal. J Biol Chem 281, 9-12

37. Sadra, A., Cinek, T. and Imboden, J. B. (2000) Multiple probing of an immunoblot membrane using a non-block technique: advantages in speed and sensitivity. Anal Biochem 278, 235-237

38. Yan, J., Kuroyanagi, H., Tomemori, T., Okazaki, N., Asato, K., Matsuda, Y., Suzuki, Y., Ohshima, Y., Mitani, S., Masuho, Y., Shirasawa, T. and Muramatsu, M. (1999) Mouse ULK2, a novel member of the UNC-51-like protein kinases: unique features of functional domains. Oncogene 18, 5850-5859

39. Tomoda, T., Bhatt, R. S., Kuroyanagi, H., Shirasawa, T. and Hatten, M. E. (1999) A mouse serine/threonine kinase homologous to C. elegans UNC51 functions in parallel fiber formation of cerebellar granule neurons. Neuron 24, 833-846

40. Robbins, D. J., Nybakken, K. E., Kobayashi, R., Sisson, J. C., Bishop, J. M. and Therond, P. P. (1997) Hedgehog elicits signal transduction by means of a large complex containing the kinesin-related protein costal2. Cell 90, 225-234

41. Sisson, J. C., Ho, K. S., Suyama, K. and Scott, M. P. (1997) Costal2, a novel kinesin-related protein in the Hedgehog signaling pathway. Cell 90, 235-245

42. Svard, J., Heby-Henricson, K., Persson-Lek, M., Rozell, B., Lauth, M., Bergstrom, A., Ericson, J., Toftgard, R. and Teglund, S. (2006) Genetic elimination of Suppressor of fused reveals an essential repressor function in the mammalian Hedgehog signaling pathway. Dev Cell 10, 187-197

43. Dunaeva, M., Michelson, P., Kogerman, P. and Toftgard, R. (2003) Characterization of the physical interaction of Gli proteins with SUFU proteins. J Biol Chem 278, 5116-5122

44. Fuccillo, M., Joyner, A. L. and Fishell, G. (2006) Morphogen to mitogen: the multiple roles of hedgehog signalling in vertebrate neural development. Nat Rev Neurosci 7, 772-783

45. Zhao, R. and Duncan, S. A. (2005) Embryonic development of the liver. Hepatology 41, 956-967

46. Omenetti, A. and Diehl, A. M. (2008) The adventures of sonic hedgehog in development and repair. II. Sonic hedgehog and liver development, inflammation, and cancer. Am J Physiol Gastrointest Liver Physiol 294, G595-598

47. Lai, K., Kaspar, B. K., Gage, F. H. and Schaffer, D. V. (2003) Sonic hedgehog regulates adult neural progenitor proliferation in vitro and in vivo. Nat Neurosci 6, 21-27

48. Wallace, V. A. and Raff, M. C. (1999) A role for Sonic hedgehog in axon-to astrocyte signalling in the rodent optic nerve. Development 126, 2901-2909

49. Palma, V., Lim, D. A., Dahmane, N., Sanchez, P., Brionne, T. C., Herzberg, C. D., Gitton, Y., Carleton, A., Alvarez-Buylla, A. and Ruiz i Altaba, A. (2005) Sonic hedgehog controls stem cell behavior in the postnatal and adult brain. Development 132, 335-344

50. Wang, B., Fallon, J. F. and Beachy, P. A. (2000) Hedgehog-regulated processing of Gli3 produces an anterior/posterior repressor gradient in the developing vertebrate limb. Cell 100, 423-434

51. Pan, Y., Bai, C. B., Joyner, A. L. and Wang, B. (2006) Sonic hedgehog signaling regulates Gli2 transcriptional activity by suppressing its processing and degradation. Mol Cell Biol 26, 3365-3377

52. Pan, Y., Wang, C. and Wang, B. (2009) Phosphorylation of Gli2 by protein kinase A is required for Gli2 processing and degradation and the Sonic Hedgehog-regulated mouse development. Dev Biol 326, 177-189

53. Wang, B. and Li, Y. (2006) Evidence for the direct involvement of {beta} TrCP in Gli3 protein processing. Proc Natl Acad Sci USA 103, 33-38

1. A method to activate Shh signaling pathway in mammalian cells, said method comprising a step of transfecting a mammalian cell with a vector comprising an isolated nucleic acid sequence encoding serine/threonine kinase of SEQ ID NO: 14.

2. The method of claim 1, wherein the mammalian cell is a stem cell.

3. The method of claim 1, wherein the cell a germinal cell of male testis.

4. A method to inhibit Shh signaling pathway in mammalian cells by providing a molecule inhibiting the serine/kinase activity of ULK3 protein.

5. The method of claim 4, wherein the molecule inhibiting the serine/kinase activity of ULK3 protein binds ATP-binding site of ULK3 kinase domain.

6. The method of claim 5, wherein the molecule inhibiting the serine/kinase activity of the ULK3 protein binds protein/peptide-binding site of the kinase domain.

7. The method of claim 6, wherein the molecule inhibiting the serine/kinase activity of the ULK3 protein is a pseudosubstrate designed based on SEQ ID NO: 15.

8. The method of claim 4, wherein the molecule inhibiting serine/kinase activity of ULK3 binds to the hydrophilic region in the C-terminal non-kinase domain of ULK3.

9. The method of claim 4, wherein the molecule inhibiting serine/kinase activity of ULK3 is a multifunctional inhibitor containing an active site binding moiety and a hydrophilic region binding moiety covalently connected to each other.

10. The method of claim 4, wherein the molecule inhibiting serine/kinase activity of ULK3 is cell permeable drug molecule that interferes with the ULK3 regulatory function in the Shh pathway.

11. A method to treat conditions related to Shh pathway signaling, said method comprising activation or inhibition of the serine/kinase activity of ULK3 protein.

12. The method of claim 11, wherein the serine/kinase activity is activated and the condition is related to male infertility, hair loss, or dwarfism or the serine/kinase activity is inhibited and the condition is cancer.

13. The method of claim 12, wherein the cancer is selected from a group consisting of prostate carcinoma, breast cancer, lung cancer, glioblastoma, esophaegal cancer, colorectal carcinoma, T-cell lymphoma, medulloblastoma, basal cell carcinoma.

14. An isolated amino acid sequence according to SEQ ID NO: 15, containing substrate site for ULK3 serine/threonine kinase activity.

15. A high affinity inhibitor of ULK3 serine/threonine activity binding to SEQ ID NO: 1 or SEQ NO: 15.


SEQUENCE LISTING (15 sequences)

SEQ ID NO: 1 - protein, 472 aa, Homo sapiens
  PEPTIDE (1)..(472): ULK3
MAGPGWGPPRLDGFILTERLGSGTYATVYKAYAKKDTREVVAIKCVAKKSLNKASVENLLTEIEILKGIRHPHIVQLKDFQWDSDNIYLIMEFCAGGDLSRFIHTRRILPEKVARVFMQQLASALQFLHERNISHLDLKPQNILLSSLEKPHLKLADFGFAQHMSPWDEKHVLRGSPLYMAPEMVCQRQYDARVDLWSMGVILYEALFGQPPFASRSFSELEEKIRSNRVIELPLRPLLSRDCRDLLQRLLERDPSRRISFQDFFAHPWVDLEHMPSGESLGRATALVVQAVKKDQEGDSAAALSLYCKALDFFVPALHYEVDAQRKEAIKAKVGQYVSRAEELKAIVSSSNQALLRQGTSARDLLREMARDKPRLLAALEVASAAMAKEEAAGGEQDALDLYQHSLGELLLLLAAEPPGRRRELLHTEVQNLMARAEYLKEQVKMRESRWEADTLDKEGLSESVRSSCTLQ

SEQ ID NO: 2 - protein, 1106 aa, Homo sapiens
  PEPTIDE (1)..(1106): GLI1
MFNSMTPPPISSYGEPCCLRPLPSQGAPSVGTEGLSGPPFCHQANLMSGPHSYGPARETNSCTEGPLFSSPRSAVKLTKKRALSISPLSDASLDLQTVIRTSPSSLVAFINSRCTSPGGSYGHLSIGTMSPSLGFPAQMNHQKGPSPSFGVQPCGPHDSARGGMIPHPQSRGPFPTCQLKSELDMLVGKCREEPLEGDMSSPNSTGIQDPLLGMLDGREDLEREEKREPESVYETDCRWDGCSQEFDSQEQLVHHINSEHIHGERKEFVCHWGGCSRELRPFKAQYMLVVHMRRHTGEKPHKCTFEGCRKSYSRLENLKTHLRSHTGEKPYMCEHEGCSKAFSNASDRAKHQNRTHSNEKPYVCKLPGCTKRYTDPSSLRKHVKTVHGPDAHVTKRHRGDGPLPRAPSISTVEPKREREGGPIREESRLTVPEGAMKPQPSPGAQSSCSSDHSPAGSAANTDSGVEMTGNAGGSTEDLSSLDEGPCIAGTGLSTLRRLENLRLDQLHQLRPIGTRGLKLPSLSHTGTTVSRRVGPPVSLERRSSSSSSISSAYTVSRRSSLASPFPPGSPPENGASSLPGLMPAQHYLLRARYASARGGGTSPTAASSLDRIGGLPMPPWRSRAEYPGYNPNAGVTRRASDPAQAADRPAPARVQRFKSLGCVHTPPTVAGGGQNFDPYLPTSVYSPQPPSITENAAMDARGLQEEPEVGTSMVGSGLNPYMDFPPTDTLGYGGPEGAAAEPYGARGPGSLPLGPGPPTNYGPNPCPQQASYPDPTQETWGEFPSHSGLYPGPKALGGTYSQCPRLEHYGQVQVKPEQGCPVGSDSTGLAPCLNAHPSEGPPHPQPLFSHYPQPSPPQYLQSGPYTQPPPDYLPSEPRPCLDFDSPTHSTGQLKAQLVCNYVQSQQELLWEGGGREDAPAQEPSYQSPKFLGDSQVSPSRAKAPVNTYGPGFGPNLPNHKSGSYPTPSPCHENFVVGANRASHRAAAPPRLLPPLPTCYGPLKVGGTNPSCGHPEVGRLGGGPALYPPPEGQVCNPLDSLDLDNTQLDFVAILDEPQGLSPPPSHDQRGSSGHTPPPSGPPNMAVGNMSVLLRSLPGETQFLNSSA

SEQ ID NO: 3 - DNA, 18 bp, artificial sequence
  chemically synthesized
aatggcggggcccggctg

SEQ ID NO: 4 - DNA, 20 bp, artificial sequence
  chemically synthesized
tctgctccagatggctcaca

SEQ ID NO: 5 - DNA, 1463 bp, Homo sapiens
  cDNA (1)..(1463): ULK3 cDNA
aatggcggggcccggctggggtcccccgcgcctggacggcttcatcctcaccgagcgcctgggcagcggcacgtacgccacggtgtacaaggcctacgccaagaaggacactcgtgaagtggtagccataaagtgtgtagccaagaaaagtctgaacaaggcatcggtggagaacctcctcacggagattgagatcctcaagggcattcgacatccccacattgtgcagctgaaagactttcagtgggacagtgacaatatctacctcatcatggagttttgcgcagggggcgacctgtctcgcttcatccatacccgcaggattctgcctgagaaggtggcgcgtgtcttcatgcagcaattagctagcgccctgcaattcctgcatgaacggaatatctctcacctggatctgaagccacagaacattctactgagctccttggagaagccccacctaaaactggcagactttggtttcgcacaacacatgtccccgtgggatgagaagcacgtgctccgtggctcccccctctacatggcccccgagatggtgtgccagcggcagtatgacgcccgcgtggacctctggtccatgggggtcatcctgtatgaagccctcttcgggcagcccccctttgcctccaggtcgttctcggagctggaagagaagatccgtagcaaccgggtcatcgagctccccttgcggcccctgctctcccgagactgccgggacctactgcagcggctcctggagcgggaccccagccgtcgcatctccttccaggacttctttgcgcacccctgggtggacctggagcacatgcccagtggggagagtctggggcgagcaaccgccctggtggtgcaggctgtgaagaaagaccaggagggggattcagcagccgccttatcactctactgcaaggctctggacttctttgtacctgccctgcactatgaagtggatgcccagcggaaggaggcaattaaggcaaaggtggggcagtacgtgtcccgggctgaggagctcaaggccatcgtctcctcttccaatcaggccctgctgaggcaggggacctctgcccgagacctgctcagagagatggcccgggacaagccacgcctcctagctgccctggaagtggcttcagctgccatggccaaggaggaggccgccggcggggagcaggatgccctggacctgtaccagcacagcctgggggagctactgctgttgctggcagcggagcccccgggccggaggcgggagctgcttcacactgaggttcagaacctcatggcccgagctgaatacttgaaggagcaggtcaagatgagggaatctcgctgggaagctgacaccctggacaaagagggactgtcggaatctgttcgtagctcttgcacccttcagtgaccctagaagaatgattggacagatgtgagccatctggagcaga

SEQ ID NO: 6 - protein, 472 aa, artificial sequence
  chemically synthesized
MAGPGWGPPRLDGFILTERLGSGTYATVYKAYAKKDTREVVAIRCVAKKSLNKASVENLLTEIEILKGIRHPHIVQLKDFQWDSDNIYLIMEFCAGGDLSRFIHTRRILPEKVARVFMQQLASALQFLHERNISHLDLKPQNILLSSLEKPHLKLADFGFAQHMSPWDEKHVLRGSPLYMAPEMVCQRQYDARVDLWSMGVILYEALFGQPPFASRSFSELEEKIRSNRVIELPLRPLLSRDCRDLLQRLLERDPSRRISFQDFFAHPWVDLEHMPSGESLGRATALVVQAVKKDQEGDSAAALSLYCKALDFFVPALHYEVDAQRKEAIKAKVGQYVSRAEELKAIVSSSNQALLRQGTSARDLLREMARDKPRLLAALEVASAAMAKEEAAGGEQDALDLYQHSLGELLLLLAAEPPGRRRELLHTEVQNLMARAEYLKEQVKMRESRWEADTLDKEGLSESVRSSCTLQ

SEQ ID NO: 7 - protein, 472 aa, artificial sequence
  MUTAGEN (1)..(472): mutated ULK3 protein ULK3 (K139R)
MAGPGWGPPRLDGFILTERLGSGTYATVYKAYAKKDTREVVAIKCVAKKSLNKASVENLLTEIEILKGIRHPHIVQLKDFQWDSDNIYLIMEFCAGGDLSRFIHTRRILPEKVARVFMQQLASALQFLHERNISHLDLRPQNILLSSLEKPHLKLADFGFAQHMSPWDEKHVLRGSPLYMAPEMVCQRQYDARVDLWSMGVILYEALFGQPPFASRSFSELEEKIRSNRVIELPLRPLLSRDCRDLLQRLLERDPSRRISFQDFFAHPWVDLEHMPSGESLGRATALVVQAVKKDQEGDSAAALSLYCKALDFFVPALHYEVDAQRKEAIKAKVGQYVSRAEELKAIVSSSNQALLRQGTSARDLLREMARDKPRLLAALEVASAAMAKEEAAGGEQDALDLYQHSLGELLLLLAAEPPGRRRELLHTEVQNLMARAEYLKEQVKMRESRWEADTLDKEGLSESVRSSCTLQ

SEQ ID NO: 8 - DNA, 20 bp, artificial sequence
  chemically synthesized
aaggagcaggtcaagatgag

SEQ ID NO: 9 - DNA, 20 bp, artificial sequence
  chemically synthesized
gtgcaagagctacgaacaga

SEQ ID NO: 10 - DNA, 21 bp, artificial sequence
  chemically synthesized
gatgatgaaccaggttatgac

SEQ ID NO: 11 - DNA, 22 bp, artificial sequence
  chemically synthesized
gtccttttcaccagcaagcttg

SEQ ID NO: 12 - DNA, 19 bp, artificial sequence
  chemically synthesized
ccttcagcaatgccagtga

SEQ ID NO: 13 - DNA, 20 bp, artificial sequence
  chemically synthesized
ctaggatctgtatagcgttt

SEQ ID NO: 14 - protein, 257 aa, Homo sapiens
  DOMAIN (1)..(257): serine/threonine kinase domain of ULK3
FILTERLGSGTYATVYKAYAKKDTREVVAIKCVAKKSLNKASVENLLTEIEILKGIRHPHIVQLKDFQWDSDNIYLIMEFCAGGDLSRFIHTRRILPEKVARVFMQQLASALQFLHERNISHLDLKPQNILLSSLEKPHLKLADFGFAQHMSPWDEKHVLRGSPLYMAPEMVCQRQYDARVDLWSMGVILYEALFGQPPFASRSFSELEEKIRSNRVIELPLRPLLSRDCRDLLQRLLERDPSRRISFQDFFAHPWV

SEQ ID NO: 15 - protein, 20 aa, artificial sequence
  chemically synthesized
SGSYPTPSPCHENFVVGANR